(19) United States
(12) Patent Application Publication
(10) Pub. No.: US 2009/0248062 A1
(43) Pub. Date: Oct. 1, 2009

(54) Device Suitable to Create A Pressure on Specific Points of the Skin

(76) Inventor: Duilio Benigna, Capriasca (CH)

Correspondence Address: NOTARO & MICHALOS P.C., 100 DUTCH HILL ROAD, SUITE 110, ORANGEBURG, NY, 10962-2100, US

(21) Appl. No.: 12/057,870

(22) Filed: Mar. 28, 2008

(51) Int. Cl. A61B 17/00

(52) U.S. Cl. 606/204

(57) ABSTRACT

A device suitable to exert pressure on specific points of the body, comprising:
a plaster applied on a support consisting of a glossy sheet suitable to make the detachment thereof easier, the said sheet being provided with a carving suitable to uncover an adhesive part of the plaster before the said plaster being removed from the support;
a pressure element consisting of a button comprising a base having the same shape as the carving made in the plaster and a protruding nipple suitable to exert a compressing action when it is applied on the skin, the said button remaining adherent to the plaster when it is removed from the support sheet.

This invention relates to a device suitable to exert pressure on specific points of the skin, consisting of a plaster removably applied on a support consisting of a glossy sheet suitable to make the detachment thereof easier, the said support being provided with a carving, which enables an adhesive part of the plaster to be uncovered before the said plaster being removed, and of a button comprising a base having the same shape as the carving in the support and a protruding nipple suitable to be compressed when it is applied to the skin, the said button remaining adherent to the plaster when the latter is removed from the support.

The result of the foregoing is a device, which offers the twofold advantage of an easy application on any point of the skin and, which has a characteristic of practicalness as a result of the easy way to apply the button, which represents the pressure element, in a particularly conformable manner, without the risk of coming into contact with the plaster adhesive portion, and with the disadvantages, which may derive therefrom.

In addition to the foregoing, the peculiar characteristics of shape and deformability of the pressure element enable to obtain a considerable effectiveness of the acupressure stimulus, which enable to effectively use the device also for applications, which usually require acupuncture.

The possibility to obtain some advantages by exerting a light pressure on specific parts of the body where specific nerve endings are located is also known.

It is therefore known that the application of a light pressure on some areas of the human body results in, may be due to the action of the massage exercised by the pressure element, a relaxing effect.

It is likewise known that it is possible to obtain, on many persons, elimination of headache or other disturbs by exerting pressure on some areas of the body, as well as it is known that by applying a bracelet, which exerts pressure on some areas of the wrist, many persons stops getting carsick.

For this reason, devices, which press the skin on specific points, which devices usually consist of bracelets fitted with a protruding element, which presses the part concerned, have been used for a long time.

These devices have however some limitations, in view of the fact that it is unlikely that a bracelet remains exactly in the same position as it is fitted, unless it is tightly tightened, with the consequent risk of a limited effectiveness or a discomfort for the user.

In addition to the foregoing, there exist parts of the body, which are sensitive to pressure, wherein it is not possible to apply a pressure element by a bracelet.

In other cases, it is possible to use a pressure element to stimulate parts of the body in relation to which the acupuncture technique is used. For example, there exists a point identified as H7 that, according to the Chinese medicine principles, improves the state of sleep, particularly suitable for those who suffer from insomnia.

A large number of patients, in this case, apply to the acupuncture technique, where the needle, acting on this point, stimulates the improvement of the state of the person. A system, which is parallelly effective is represented by acupressure, that is a system acting with a punctiform pressure on the point itself.

On the other hand, it is clear that the results depend much also on the presser characteristics, which presser must be able to exert a correct pressure without, however, disturbing the person, gently acting on the part to be pressed, where such presser is kept also for a long time.

The present invention seeks to overcome these drawbacks, by proposing a device suitable to exert pressure on specific points of the skin, consisting of a plaster, which can be easily applied to any part of the body and that is provided with a pressure element consisting of a button provided with a nipple or, however, with a protruding element made of soft material, which exerts pressure on the desired point.

According to the invention, the plaster is applied on a so-called liner, or a support provided with a glossy surface, which makes the plaster easy to be removed and that is provided with a circular carving in the position that is just close to the area wherein the button must be fixed, which button can be so applied to the plaster when the entire remaining adhesive part is protected by liner, thus making the operation particularly easy without the risk of coming into contact with the adhesive parts.

The present invention will now be more particularly described, by way of example only, with reference to the accompanying figures, in which:

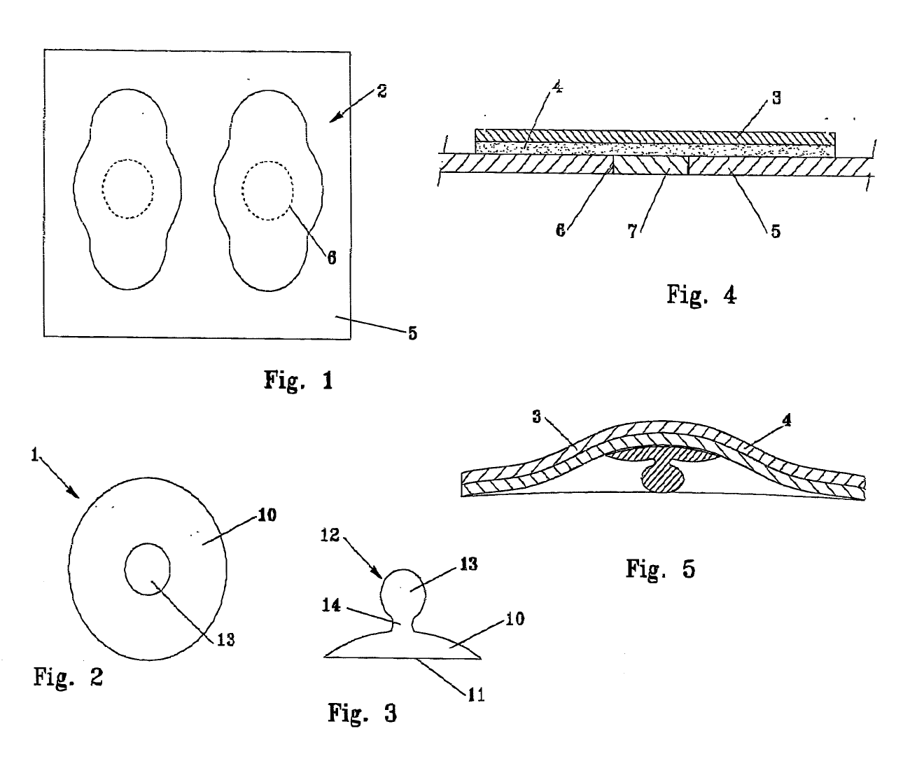

FIG. 1 is top view of a typical package of the product, consisting of a liner on which two plasters ready for application on the pressure element have been applied;

FIGS. 2 and 3 are the top view and the side view of the pressure element respectively;

FIG. 4 is perspective view of a device according to the invention ready for use;

FIG. 5 schematically shows a device according to the invention, applied to the skin.

With reference to the figures annexed hereto, the device according to the invention consists of a pressure element indicated, as a whole, with reference number 1 and a plaster indicated as a whole, with reference number 2, to which the pressure element is fixed and that allows to subsequently apply such element to the skin on the desired point. The plaster is multilayered, with a first layer 3 consisting of a primary support made of textile material, in particular a non-woven fabric made of Viscose (registered trade-mark) made according to Jet Laced technology. It is a fabric resulting from the action of hundreds jets of water under pressure, which interlace the fibres and that has a characteristic of considerable importance for this particular application, since, beside the total absence of any chemical bond that may cause allergic reactions as a result of contact with skin, has a percentage elongation between warp and weft, which elongation reaches values of 40% as to warp and 60% as to weft. Such characteristic allows the natural bending of the plaster support, which, accordingly, can follow the skin movements, for example the movements of hand, while keeping a perfect adhesion and minimising the effects due to irritation.

This layer of support is coated by a second layer 4 made of self-adhesive material, consisting of dermocompatible acrylic polymers of a known type. To the adhesive material, an amount of additives and active ingredients, always of a known type, is added, in such a way as to prevent any possible allergic reactions to the daily use of the device.

A preferred version of the invention provides for specific oils, of a known type too, which make the following detachment of the plaster from the skin easier.

The plaster according to the description/above is applied on a so-called liner, that is a sheet 5 of monoglossy paper coated on a side with a silicone material layer, which has a glossy surface suitable to make the detachment thereof easier.

The liner is provided, by punching, with a carving 6 just close to the central area of the plaster which has the same shape and the same size as the base of the pressure element.

Such shape 7, once it has been removed, uncovers a portion of adhesive surface of the plaster, thus allowing the application of the base of the pressure element, while keeping the remaining adhesive surface covered and protected and preventing it from sticking to the fingers during the operation.

In addition to the foregoing, a preferred version of the invention may provide for more than one plaster, which may be taken out one at a time. The button that constitutes the pressure element 1, has a thin and flexible base 10, in the case shown having a rounded shape, with a plane lower surface 11 and a nipple 12, consisting of a rounded body 13 connected to the base 10 by a portion 14 having a reduced diameter.

The said pressure element, preferably obtained by injection, is made of PVC, by using material having a hardness ranging between 50 and 100 Shores, preferably equal to 75 Shores, which hardness turns out to be optimal for creating the desired effect without disturbing the point where the pressure is exerted, since the material results to IDS sufficiently deformable even though it exerts the requested action.

In order to make the device ready, the user does nothing but removes the punched shape 7 from one of the plasters, applies a pressure element by fitting the base into the area inside the carving 6 of the plaster, until it rests on the uncovered surface of the adhesive.

The plaster, to which the presser will be applied, protruding from the portion of the adhesive surface, can be now removed from the liner.

Now the application results to be extremely simple, in view of the fact that the presser must be simply located just closes to the area concerned and then fixed in position by applying the plaster to the skin, which plaster will keep the nipple made of deformable material perfectly positioned.

The plaster, thanks to the elasticity characteristics of the non-woven fabric, may follow the movements of the hand without detaching from it.

A device, which is considerably practical and comfortable, which can be quickly prepared and applied with the same practicalness is thus obtained.

1. Device suitable to exert pressure on specific points of the skin, characterised in that it provides for:
a plaster applied on a support consisting of a glossy sheet suitable to make the detachment thereof easier, the said sheet being provided with a carving suitable to uncover an adhesive part of the plaster before the said plaster being removed from the support;
a pressure element consisting of a button comprising a base having the same shape as the carving made in the plaster and a protruding nipple suitable to exert a compressing action when it is applied on the skin, the said button remaining adherent to the plaster when it is removed from the support sheet.

2. Device according to claim 1, wherein the said carving is made just close to the central area of the plaster.

3. Device according to claim 1, wherein the pressure element is made of a material having a hardness ranging from 50 and 100 Shores.

4. Device according to claim 3, wherein the pressure element is made of a material having a hardness equal to 76 Shores.

5. Device according to claim 1, wherein the pressure element is made of PVC.

6. Device according to claim 1, characterised in that the plaster is made of non-woven fabric coated with an adhesive material layer.

7. Device according to claim 6, characterised in that the said non-woven fabric is made of viscose.

8. Device according to claim 7, wherein the said viscose fabric is made according to Jet Laced technology.

9. Device according to claim 1, characterised in that the said adhesive is mixed with anergic additives.
